(19) United States
(12) Patent Application Publication
(10) Pub. No.: US 2026/0090738 A1
(43) Pub. Date: Apr. 2, 2026

(54) SITTING POSTURE CORRECTION CHAIR WITH REAL-TIME MONITORING AND REMINDER FUNCTIONS

(71) Applicant: GUANGZHOU LIUQUAN BRAND MANAGEMENT SERVICE CO., LTD., Guangzhou City (CN)

(72) Inventor: Libin Chen, Fuqing City (CN)

(21) Appl. No.: 18/914,774

(22) Filed: Oct. 14, 2024

(30) Foreign Application Priority Data
Sep. 29, 2024 (CN) 202422385000.X

(51) Int. Cl. A61B 5/11; A61B 5/00

(52) U.S. Cl. CPC A61B 5/1126; A61B 5/6891

(57) ABSTRACT

Disclosed is a sitting posture correction chair with real-time monitoring and reminder functions. The sitting posture correction chair includes a main body component, and further includes a sitting posture monitoring and warning module. The sitting posture monitoring and warning module is sleeved externally on a back of the main body component. The sitting posture monitoring and warning module is capable of monitoring data of pressure endured by a plurality of areas on a back of the main body component. The sitting posture monitoring and warning module is capable of emitting a prompt sound. The sitting posture correction chair with real-time monitoring and reminder functions not only overcomes many defects of sitting posture correction devices in the prior art, but also provides users with a more intelligent, comfortable, and convenient sitting posture correction solution through a series of innovative designs, demonstrating significant technological advancement and broad application prospects.

### TECHNICAL FIELD

The present disclosure relates to the technical field of sitting posture chairs, and specifically relates to a sitting posture correction chair with real-time monitoring and reminder functions.

### BACKGROUND

With the accelerating pace of modern life and changes in work styles, it has become normal for people to sit on office chairs or study chairs for a long time. However, improper sitting habits usually lead to uneven distribution of pressure on spines of users, and even cause health issues such as lumbar disc herniation and cervical spondylosis, which will severely affect their life quality and work efficiency. Therefore, how to effectively monitor and correct sitting postures has become a topic of great concern.

Conventional sitting posture correction methods rely much on individual consciousness or supervision by others, thereby having defects of strong subjectivity and supervision instability. With rapid development of sensor technology, IoT technology, and intelligent algorithms in recent years, new technological approaches are developed for sitting posture correction. Although some sensor-based sitting posture monitoring devices have appeared on the market, most of them have defects such as monitoring area limitation, slow response, poor user experience and the like, and hardly reflect users’ sitting postures in a comprehensive, real-time and accurate manner.

### SUMMARY

In view of the defects in the prior art, the present disclosure provides a sitting posture correction chair with real-time monitoring and reminder functions, to solve the technical problems mentioned in the above Background.

In order to achieve the above objective, the present disclosure is achieved by the following technical solution: a sitting posture correction chair with real-time monitoring and reminder functions, including a main body component, where an upper part of the main body component is sleeved with a sitting posture monitoring and warning module, the sitting posture monitoring and warning module is provided with sensors, the sitting posture monitoring and warning module is capable of performing real-time monitoring on a plurality of areas on a back of the main body component through the sensors, and the sitting posture monitoring and warning module is capable of emitting a prompt sound to remind a user to maintain a proper sitting posture.

Further, the sitting posture monitoring and warning module includes a cloth cover sleeved externally on the back of the main body component, the cloth cover has a double-layer structure on one side, and sensors are symmetrically fixed in the double-layer structure; a warning device is fixed to a back of the cloth cover, and power supply and communication between the warning device and the sensors are achieved through a wire harness.

Further, the cloth cover is made of non-woven fabric, the sensors include a piezoelectric film sensor, a pressure sensor or a capacitive sensor, the wire harness includes a power supply wire and a communication wire, a bottom end of the cloth cover is provided with an opening for externally sleeving on the back of the main body component, second binding straps are symmetrically connected at the opening, one end of either of the second binding straps is fixedly connected to the cloth cover, and the other end thereof is connected to the cloth cover through a button or a hook-and-loop fastener.

Further, the cloth cover is divided into a first monitoring area and a second monitoring area, the sensors are correspondingly fixed inside the first monitoring area and the second monitoring area, and a second hollow part shaped like a character “U” or “V” is arranged between the first monitoring area and the second monitoring area.

Further, the warning device includes a housing body fixed on the back of the cloth cover, a power supply and a circuit board are respectively fixed inside the housing body, and a speaker is mounted at an outer end of the housing body in a snap-fitted manner.

Further, the circuit board is integrated with a charging interface and a contact switch respectively, edges of the housing body are provided with a reserved port I and a reserved port II, a charging end of the charging interface is exposed outside the housing body through the reserved port I, and a switch button is movably arranged inside the reserved port II, where the switch button corresponds to the contact switch in position.

Further, the main body component is provided with a frame as a basic supporting structure, the frame is a curved component, and the frame includes a chair back, a chair seat and a lumbar support, where the chair back, the chair seat and the lumbar support form an integrated structure, and the lumbar support is arranged between the chair back and the chair seat in a connected manner; a middle portion of the chair back is reservedly provided with a first hollow part that fits with the second hollow part; and a plurality of snapping holes are formed in interiors of the chair back and the chair seat, and the ventilation holes are symmetrically formed inside the lumbar support.

Further, backup plates are symmetrically mounted on a surface of the chair back, a back cushion is adhesively fixed on a surface of either of the backup plates, seat boards are symmetrically mounted on a surface of the chair seat, and seat cushions are adhesively fixed on surfaces of the seat boards.

Further, a plurality of through holes are formed in interiors of the backup plates and the seat boards, the through holes correspond to the snapping holes in positions, the chair back and the backup plates, as well as the chair seat and the seat boards, are snap-fitted and fixed through a plurality of snap latches, and the snap latches penetrate through the through holes and are snap-fitted and fixed with the snapping holes.

Further, a top end of either of the back cushions is connected to a first binding strap, the snapping holes are formed at one end of the first binding strap opposite to the corresponding back cushion, the first binding strap is wrapped from a front side of the chair back to a back side of the chair back, and the snapping holes and the snap latches are snap-fitted and fixed.

The present disclosure provides a sitting posture correction chair with real-time monitoring and reminder functions. Through an ingenious combination of the sitting posture monitoring and warning module with the main body component, the present disclosure achieves accurate, real-time monitoring and timely correction of users’ sitting postures. Significant beneficial effects are mainly reflected in the following aspects:

1.Precise monitoring and instant feedback: the piezoelectric film sensors are selected as the sensors due to compactness in thickness and size and can be precisely placed in different areas of the chair back, such that a pressure and position of a user’s back leaning against the chair back can be comprehensively monitored. Once any poor sitting posture is detected, e.g., when the user’s back is not in full contact with the chair back or the pressure of leaning against the chair back is uneven, the sitting posture monitoring and warning module is capable of immediately emitting a prompt sound to remind the user to adjust the posture in a timely manner, thereby effectively avoiding subjectivity and hysteresis of conventional sitting posture correction methods.

2. Comfort and protection: a double-layer structure design of the cloth cover and arrangement of the second hollow part corresponding to the first hollow part of the chair back ensure effective installation and operation of the sensors and prevent direct contact between a user’s spine and the chair back, thereby reducing spinal pressure, offering a more comfortable sitting experience, and protecting the user’s spine.

3.Intelligence and convenience: the sitting posture monitoring and warning module integrates the components such as the power supply, the circuit board and the speaker, and is capable of easily activating and deactivating the entire system through the simple contact switch. A design of the charging interface enables repeated recharging of the power supply, and extends a service life of the chair. Further, use of wire harnesses achieves power supply and communication and ensures stable signal transmission.

4. Modular design and easy maintenance: the modular design of the main body component enables fast assembly and disassembly of the components such as the back cushions and the seat cushions from the frame through the snap latches, which not only facilitates replacement of cushions of different materials by users according to their personal preferences, but also greatly facilitates routine cleaning and maintenance, such as disassembly for cleaning or replacement of worn-out components.

5. Health promotion and efficiency improvement: long-term proper use of the sitting posture correction chair of the present disclosure can significantly optimize users’ sitting habits, reduce health issues caused by poor sitting posture, such as spinal diseases and cervical spondylosis, thereby enhancing the users’ quality of life. Moreover, good sitting posture helps to stayed focused and improve work efficiency, and the present disclosure is particularly suitable for office workers and students who need to sit for a long time.

To sum up, the present disclosure not only overcomes many defects of sitting posture correction devices in the prior art, but also provides users with a more intelligent, comfortable, and convenient sitting posture correction solution through a series of innovative designs, demonstrating significant technological advancement and broad application prospects.

### BRIEF DESCRIPTION OF THE DRAWINGS

FIG. 1 is a schematic diagram of a breakdown structure of the present disclosure.

FIG. 2 is a schematic diagram of a breakdown structure of a sitting posture monitoring and warning module of the present disclosure.

FIG. 3 is a structural schematic diagram of a first view of the present disclosure after assembly of a sitting posture monitoring and warning module.

FIG. 4 is a structural schematic diagram of a second view of the present disclosure after assembly of a sitting posture monitoring and warning module.

FIG. 5 is a schematic diagram of a breakdown structure of a warning device of the present disclosure.

FIG. 6 is a schematic diagram of a breakdown structure of a main body component of the present disclosure.

FIG. 7 is a structural schematic diagram of a frame of the present disclosure.

FIG. 8 is a structural schematic diagram of a back cushion of the present disclosure.

In the figures: 1. main body component;11. frame; 111. chair back; 112. chair seat; 113. lumbar support; 114. first hollow part; 115. snapping hole; 116. ventilation hole; 12. backup plate; 13. back cushion; 131. first binding strap; 14. seat board; 15. seat cushion; 16. through hole; 17.snap latch; 2. sitting posture monitoring and warning module; 21. cloth cover; 211. first monitoring area; 212. second monitoring area; 213. second hollow part; 22. sensor; 23. warning device; 231. housing body; 232. switch button; 233. power supply; 234. circuit board; 235. speaker; 236. charging interface; 24. wire harness; and 25. second binding strap.

### DETAILED DESCRIPTIONS OF THE EMBODIMENTS

The technical solutions in the embodiments of the present disclosure will be clearly and completely described below in combination with the accompanying drawings in the embodiments of the present disclosure. Apparently, the embodiments described are merely some rather than all of the embodiments of the present disclosure. Based on the embodiments of the present disclosure, all other embodiments obtained by those of ordinary skill in the art without making creative efforts shall fall within the protection scope of the present disclosure.

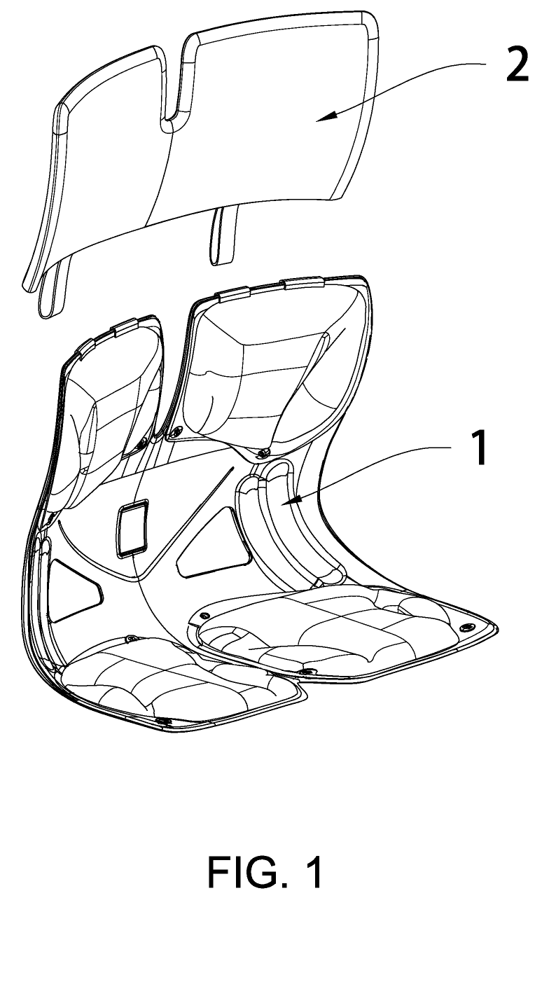

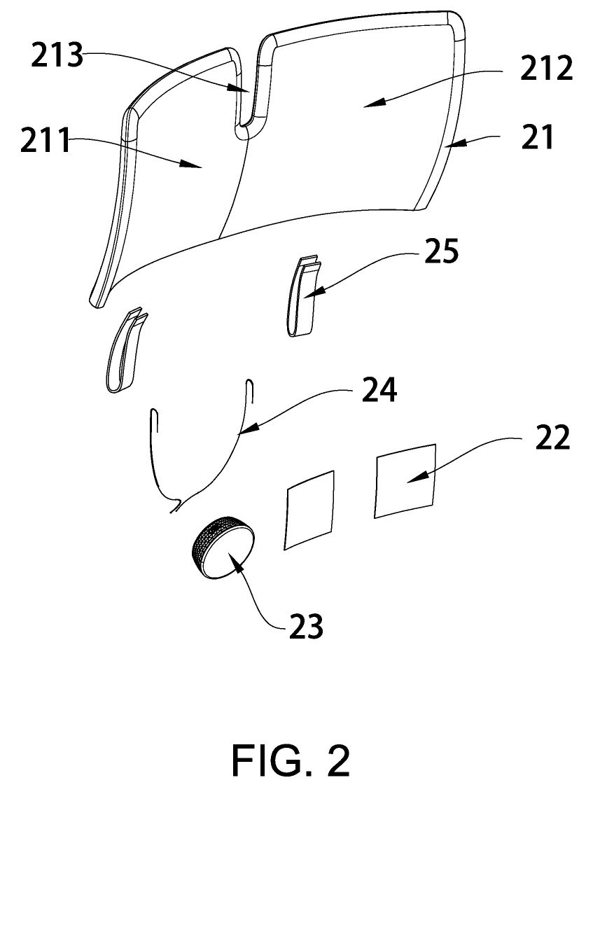

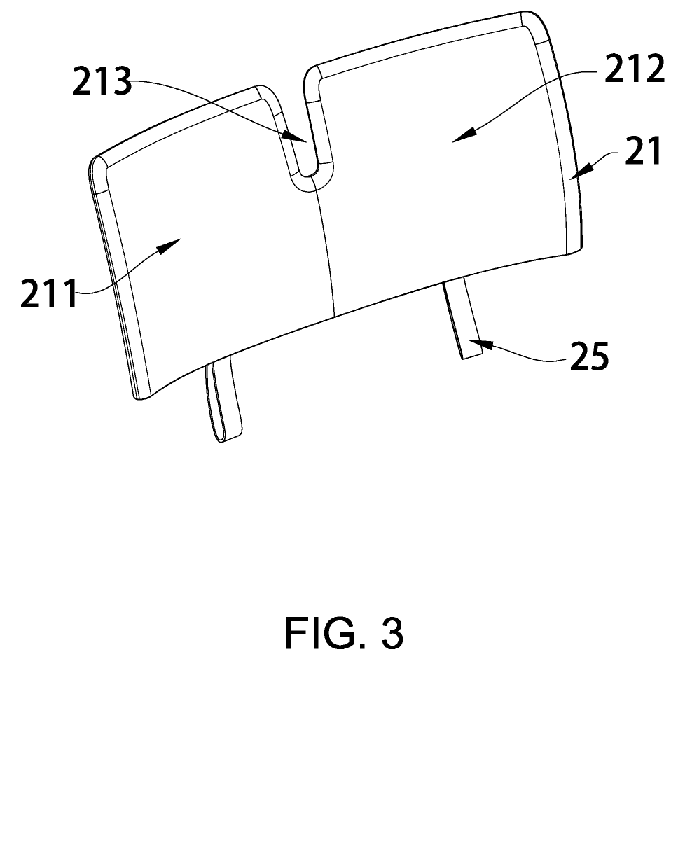

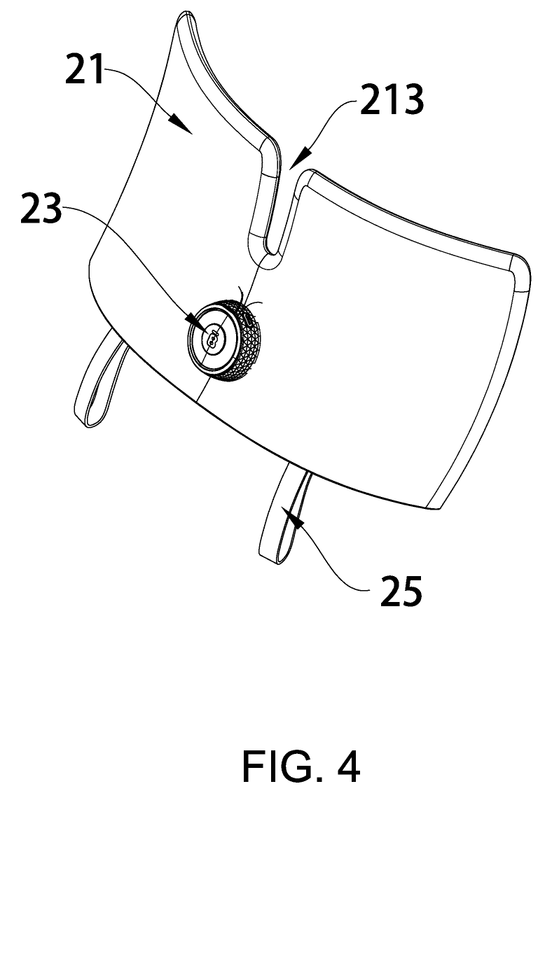

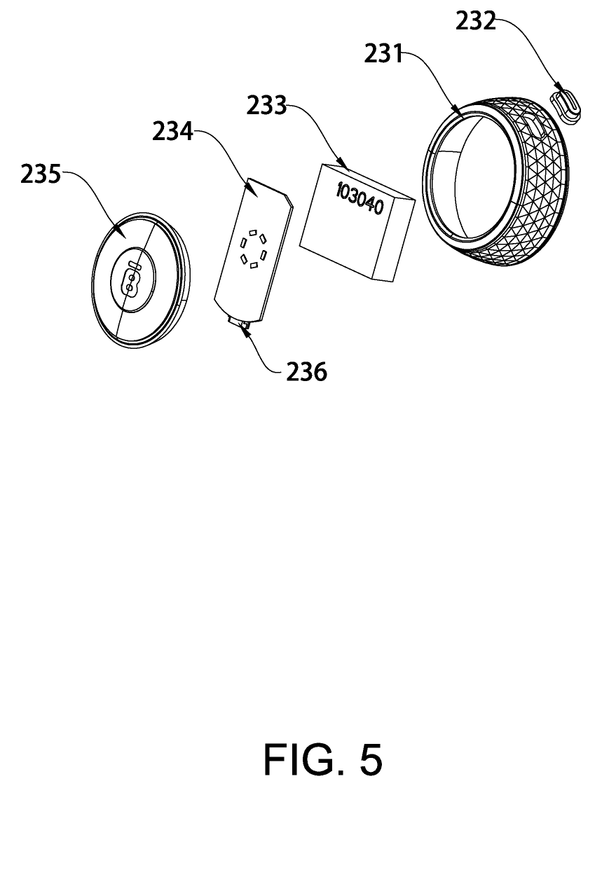

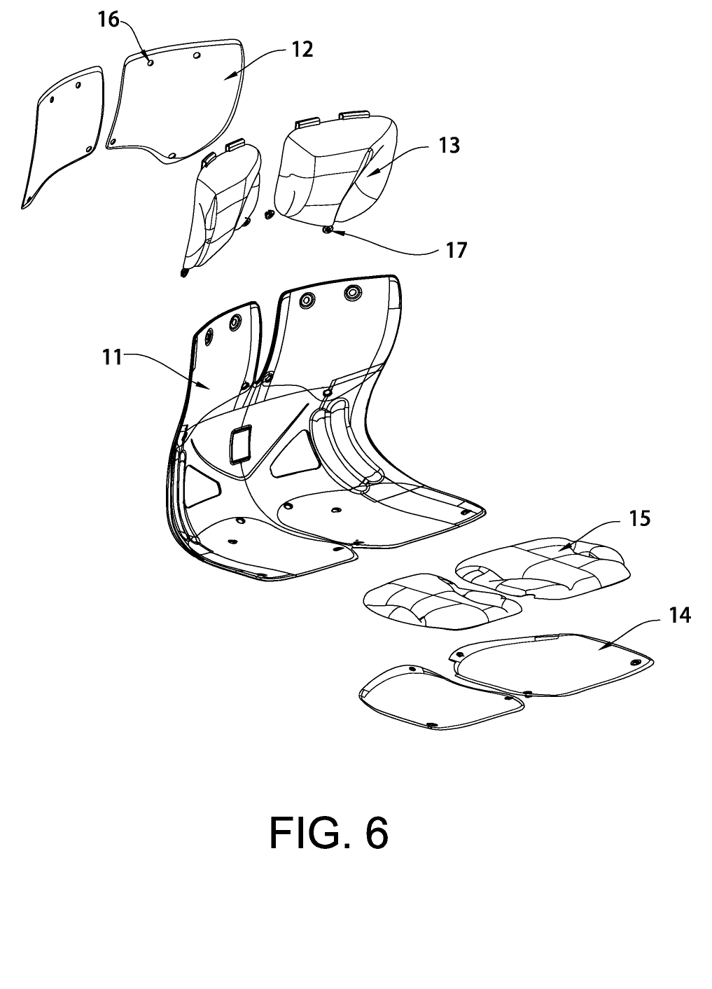

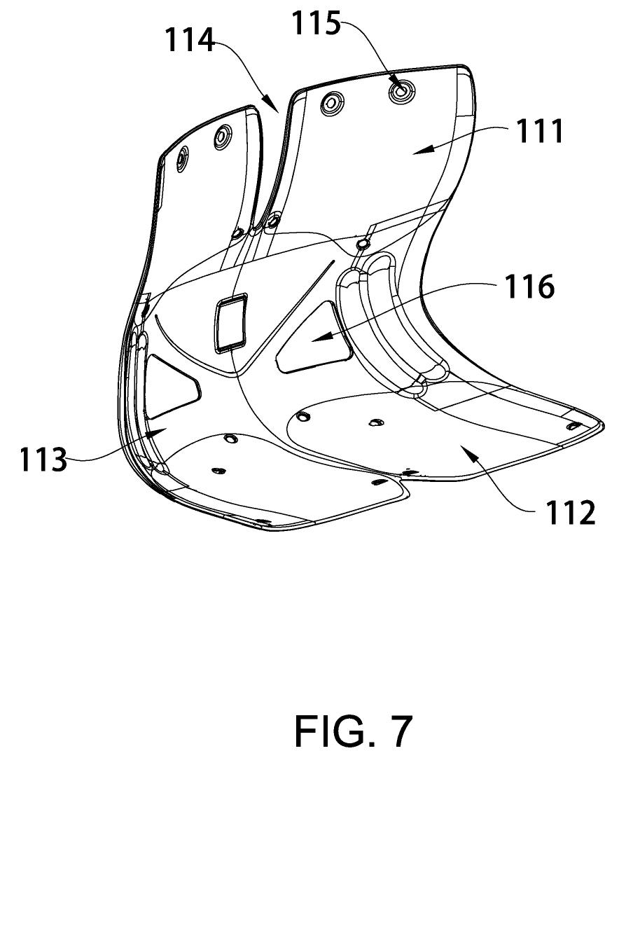

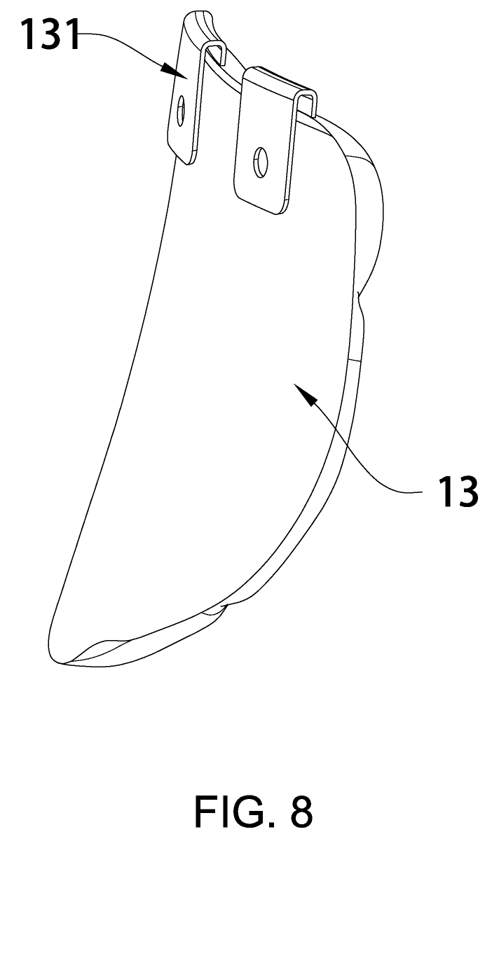

With reference to FIGS. 1-8, the present disclosure provides a technical solution: a sitting posture correction chair with real-time monitoring and reminder functions, includes a main body component 1, where an upper part of the main body component 1 is sleeved with a sitting posture monitoring and warning module 2, the sitting posture monitoring and warning module 2 is provided with sensors, the sitting posture monitoring and warning module 2 is capable of performing real-time monitoring on a plurality of areas on a back of the main body component 1 through the sensors, and the sitting posture monitoring and warning module 2 is capable of emitting a prompt sound to remind a user to maintain a proper sitting posture. For example, after a user sits on the main body component 1 and activates the sitting posture monitoring and warning module 2, when the user’s back is not leaning against the back of the main body component 1, or a pressure exerted by the user’s back is uneven, that is, one side of the user’s back is in full contact with the back of the main body component 1 while the other side thereof is separated therefrom, it is determined that the user’s sitting posture is improper. In this case, the sitting posture monitoring and warning module 2 will emit a corresponding prompt sound to remind the user to correct his/her sitting posture.

The above effect is specifically realized through the following structure:

The sitting posture monitoring and warning module 2 includes a cloth cover 21 sleeved externally on the back of the main body component 1 (the cloth cover 21 is made of non-woven fabric), the cloth cover 21 has a double-layer structure on one side, and sensors 22 are symmetrically fixed in the double-layer structure (the sensors 22 include a piezoelectric film sensor, a pressure sensor or a capacitive sensor, and these sensors are thinner and smaller in size, and are suitable for assembly into the cloth cover 21 for use. Specific structure and principles of the sensors are well-known in the prior art and will not be described in detail herein.) A warning device 23 is fixed to a back of the cloth cover 21, and power supply and communication between the warning device 23 and the sensors 22 are achieved through a wire harness 24 (i.e., the wire harness 24 includes a power supply wire and a communication wire).

A bottom end of the cloth cover 21 is provided with an opening for externally sleeving on the back of the main body component 1, and second binding straps 25 are symmetrically connected at the opening, where one end of either of the second binding straps 25 is fixedly connected to the cloth cover 21, and the other end of either of the second binding straps 25 penetrates through a ventilation hole 116 and is connected to the cloth cover 21 through a button or a hook-and-loop fastener. The cloth cover 21 is divided into a first monitoring area 211 and a second monitoring area 212 (the first monitoring area 211 and the second monitoring area 212 correspond to two sides of a human body back), and the sensors 22 are correspondingly fixed inside the first monitoring area 211 and the second monitoring area 212. A second hollow part 213 shaped like a character “U” or “V” is arranged between the first monitoring area 211 and the second monitoring area 212. Arrangement of the second hollow part 213 is capable of preventing the user’s spine from direct contact with the chair, thus avoiding pressure on the spine and achieving protection of the spine.

Further, the warning device 23 includes a housing body 231 fixed on the back of the cloth cover 21, and a power supply 233 and a circuit board 234 are respectively fixed inside the housing body 231 (the power supply 233 is capable of supplying power to the circuit board 234, and further supplying power to the sensors 22 and a speaker 235). The speaker 235 is mounted at an outer end of the housing body 231 in a snap-fitted manner (the speaker 235 is capable of broadcasting prompt sounds related to improper sitting posture according to settings). The circuit board 234 is integrated with a charging interface 236 and a contact switch respectively (The charging interface 236 facilitates charging of the power supply 233, and the contact switch facilitates activation and de activation of the sitting posture monitoring and warning module 2). Edges of the housing body 231 are provided with a reserved port I and a reserved port II, a charging end of the charging interface 236 is exposed outside the housing body 231 through the reserved port I, and a switch button 232 is movably arranged inside the reserved port II, where the switch button 232 corresponds to the contact switch in position.

Specifically, the main body component 1 is provided with a frame 11 as a basic supporting structure, the frame 11 is a curved component (that is ergonomically designed for greater comfort), and the frame 11 includes a chair back 111, a chair seat 112 and a lumbar support 113, where the chair back 111, the chair seat 112 and the lumbar support 113 form an integrated structure, and the lumbar support 113 is arranged between the chair back 111 and the chair seat 112 in a connected manner. A middle portion of the chair back 111 is reservedly provided with a first hollow part 114 that fits with the second hollow part 213 (functions of the first hollow part 114 and the second hollow part 213 are the same). A plurality of snapping holes 115 are formed in interiors of the chair back 111 and the chair seat 112, and the ventilation holes 116 are symmetrically formed inside the lumbar support 113. Backup plates 12 are symmetrically mounted on a surface of the chair back 111, and a back cushion 13 is adhesively fixed on the surface of either of the backup plates 12. Seat boards 14 are symmetrically mounted on a surface of the chair seat 112, and seat cushions 15 are adhesively fixed on surfaces of the seat boards 14. A plurality of through holes 16 are formed in interiors of the backup plates 12 and the seat boards 14, and the through holes 16 correspond to the snapping holes 115 in positions. The chair back 111 and the backup plates 12, as well as the chair seat 112 and the seat boards 14, are snap-fitted and fixed through a plurality of snap latches 17, and the snap latches 17 penetrate through the through holes 16 and are snap-fitted and fixed with the snapping holes 115. A top end of either of the back cushions 13 is connected to a first binding strap 131, the snapping holes 115 are formed at one end of the first binding strap 131 opposite to the corresponding back cushion 13, and the first binding strap 131 is wrapped from a front side of the chair back 111 to a back side of the chair back 111. The snapping holes 115 and the snap latches 17 are snap-fitted and fixed. Through the above structure, both the back cushions 13 and the seat cushions 15 can be removed from the frame 11, thereby facilitating routine maintenance of the back cushions 13 and the seat cushions 15, such as disassembly, replacement, or cleaning.

To sum up, the sitting posture correction chair with real-time monitoring and reminder functions not only overcomes many defects of sitting posture correction devices in the prior art, but also provides users with a more intelligent, comfortable, and convenient sitting posture correction solution through a series of innovative designs, demonstrating significant technological advancement and broad application prospects.

What is claimed is:

1. A sitting posture correction chair with real-time monitoring and reminder functions, comprising a main body component, wherein an upper part of the main body component is sleeved with a sitting posture monitoring and warning module, the sitting posture monitoring and warning module is provided with sensors, the sitting posture monitoring and warning module is capable of performing real-time monitoring on a plurality of areas on a back of the main body component through the sensors, and the sitting posture monitoring and warning module is capable of emitting a prompt sound to remind a user to maintain a proper sitting posture.

2. The sitting posture correction chair with real-time monitoring and reminder functions according to claim 1, wherein the sitting posture monitoring and warning module comprises a cloth cover sleeved externally on the back of the main body component, the cloth cover has a double-layer structure on one side, and sensors are symmetrically fixed in the double-layer structure; and a warning device is fixed to a back of the cloth cover, and power supply and communication between the warning device and the sensors are achieved through a wire harness.

3. The sitting posture correction chair with real-time monitoring and reminder functions according to claim 2, wherein the sensors comprise a piezoelectric film sensor, a pressure sensor or a capacitive sensor, the wire harness comprises a power supply wire and a communication wire, a bottom end of the cloth cover is provided with an opening for externally sleeving on the back of the main body component, second binding straps are symmetrically connected at the opening, one end of either of the second binding straps is fixedly connected to the cloth cover, and the other end thereof is connected to the cloth cover through a button or a hook-and-loop fastener.

4. The sitting posture correction chair with real-time monitoring and reminder functions according to claim 2, wherein the cloth cover is divided into a first monitoring area and a second monitoring area, the sensors are correspondingly fixed inside the first monitoring area and the second monitoring area, and a second hollow part shaped like a character “U” or “V” is arranged between the first monitoring area and the second monitoring area.

5. The sitting posture correction chair with real-time monitoring and reminder functions according to claim 2, wherein the warning device comprises a housing body fixed on the back of the cloth cover, a power supply and a circuit board are respectively fixed inside the housing body, and a speaker is mounted at an outer end of the housing body in a snap-fitted manner.

6. The sitting posture correction chair with real-time monitoring and reminder functions according to claim 5, wherein the circuit board is integrated with a charging interface and a contact switch respectively, edges of the housing body are provided with a reserved port I and a reserved port II, a charging end of the charging interface is exposed outside the housing body through the reserved port I, and a switch button is movably arranged inside the reserved port II, wherein the switch button corresponds to the contact switch in position.

7. The sitting posture correction chair with real-time monitoring and reminder functions according to claim 1, wherein the main body component is provided with a frame as a basic supporting structure, the frame is a curved component, and the frame comprises a chair back, a chair seat and a lumbar support, wherein the chair back, the chair seat and the lumbar support form an integrated structure, and the lumbar support is arranged between the chair back and the chair seat in a connected manner; a middle portion of the chair back is reservedly provided with a first hollow part that fits with the second hollow part; and a plurality of snapping holes are formed in interiors of the chair back and the chair seat, and the ventilation holes are symmetrically formed inside the lumbar support.

8. The sitting posture correction chair with real-time monitoring and reminder functions according to claim 7, wherein backup plates are symmetrically mounted on a surface of the chair back, a back cushion is adhesively fixed on a surface of either of the backup plates, seat boards are symmetrically mounted on a surface of the chair seat, and seat cushions are adhesively fixed on surfaces of the seat boards.

9. The sitting posture correction chair with real-time monitoring and reminder functions according to claim 8, wherein a plurality of through holes are formed in interiors of the backup plates and the seat boards, the through holes correspond to the snapping holes in positions, the chair back and the backup plates, as well as the chair seat and the seat boards, are snap-fitted and fixed through a plurality of snap latches, and the snap latches penetrate through the through holes and are snap-fitted and fixed with the snapping holes.

10. The sitting posture correction chair with real-time monitoring and reminder functions according to claim 8, wherein a top end of either of the back cushions is connected to a first binding strap, the snapping holes are formed at one end of the first binding strap opposite to the corresponding back cushion, the first binding strap is wrapped from a front side of the chair back to a back side of the chair back, and the snapping holes and the snap latches are snap-fitted and fixed.
